(19) United States
(12) Patent Application Publication
(10) Pub. No.: US 2021/0077357 A1
(43) Pub. Date: Mar. 18, 2021

(54) TUBIE VENT GASTROSTOMY VENTING UNIT

(71) Applicant: VIRGINIA COMMONWEALTH UNIVERSITY, Richmond, VA (US)

(72) Inventor: Nancy THOMPSON, Chesterfield, VA (US)

(21) Appl. No.: 17/103,245

(22) Filed: Nov. 24, 2020

Related U.S. Application Data
(62) Division of application No. 16/789,987, filed on Feb. 13, 2020.
Application No. 16/789,987 is a continuation of application No. PCT/US2019/030317, filed on May 2, 2019.
(60) Provisional application No. 62/665,551, filed on May 2, 2018.

(51) Int. Cl. A61J 15/00

(52) U.S. Cl. CPC A61J 15/0096; A61J 15/0057; A61J 15/0015; A61J 15/0003

(57) ABSTRACT

A gastrostomy or nasogastric tube venting unit has a containment vessel for measuring fluids expelled with gastric gas before, during, after, or independent from feeding. The venting unit is particularly well-suited for pediatric or extremely weak patients who have difficulty producing sufficient pressure to expel gas through a fluid-filled feeding tube, including babies weighing less than 2000 grams. The venting unit includes a handle for suspending from an IV pole, a car or infant seat, stroller, a coat hook, or other support structures that might be used for travel. The containment vessel has a splash guard in the form of mesh material or screen covering or molded within the upper opening of the vessel, and can be capped temporarily for ease of transportation. The cap can be reopened when the patient is secured in a car seat or other means for travel, so that venting can resume. The splash-guard screen prevents spilling, spraying or splashing of liquids when gastric contents are explosively expelled along with gas.

### CROSS-REFERENCE TO RELATED APPLICATIONS

This application claims benefit of U.S. Provisional Application No. 62/665,551, filed May 2, 2018, the complete contents of which is hereby incorporated by reference.

### BACKGROUND OF THE INVENTION

### Field of the Invention

The invention generally relates to a system for venting gastric pressure to relieve discomfort and gastric distress associated with various medical conditions, including feeding via gastrostomy or nasogastric feeding tube. The invention is particularly suited for venting gastric pressure in a neonate, newborn or premature infant, and children having a gastrostomy or a nasogastric feeding tube.

### Background

When patients have difficulty eating, swallowing or absorbing nutrients, short-term nutrition needs can be met with liquid feeding via a nasogastric tube passed down the esophagus to the stomach. When a longer-term solution is required, a patient may undergo surgical placement of a tube entering the stomach from outside the abdomen, known as a gastrostomy. Gastrostomy is sometimes performed to feed a patient, decompress the stomach of a patient, or to permit healing of surgical repair to prevent reflux, or other surgery. The normal build-up of gases produced in the stomach during digestion must be emitted by means other than the normal eructation or burping mechanism for relief of pressure, and this is particularly an issue to avoid damaging surgical repairs or to prevent aspiration. Gases can also build up during crying or other activity that cause intake of air, and pressure on the gastric contents is also increased by coughing, sneezing, or other actions that impinge on the abdomen. In cases where patients are also undergoing treatments that cause nausea, such as chemotherapy treatments, venting can reduce retching and/or vomiting, independent from the need for venting associated with feeding.

Venting during enteral feeding can be particularly challenging since liquid nutritional formulas are frequently expelled during venting episodes. The FARRELL® Valve System (Corpak MedSystems, Inc; Buffalo Grove, Ill.) is a bag vented to allow the escape of gas during enteral feeding. See also US 2016/0067148, which discloses the apparatus of the FARRELL® Valve System. However, the FARRELL® bag can be cost prohibitive for prolonged usage and only solves a portion of the problems associated with venting. Although US 2026/0067148 teaches pediatric applications, the FARRELL® bag has been found difficult if not impossible to use with very small newborns or premature infants. This is because the FARRELL® bag requires significant pressure for fluid to be pushed into the bag and infants under 2000 grams are typically unable to exert sufficient pressure. In an attempt to solve this problem, pediatric and neonatal care nurses frequently use a 2 ounce syringe connected to tubing that further connects to a delivery tube, and allow liquid food to drip slowly into the syringe. The slow drip of liquid allows simultaneous venting through the delivery tube. However, this makeshift design is cumbersome since the liquid frequently bubbles out, spills, and may become contaminated with debris that can fall into the syringe. Loss of liquid makes it more difficult to determine nutritional intake, and this is particularly a problem when caring for neonates/newborns under 2000 grams, since these small babies are particularly vulnerable to errors arising from miscalculation of nutritional intake.

The same issue of inadequate pressure to achieve venting occurs with children and extremely sick or weak adult patients. Furthermore, the difficulties of venting and feeding create a situation wherein patients discharged from the hospital still remain home-bound and caregiving is more expensive and difficult. Proper venting contributes to the success of enteral feeding, which aims to avoid body weight loss, correct significant nutritional deficiencies, promote growth in children, and improve quality of life for patients and caregivers. A self-contained and portable solution to allow venting as needed, retain nutritional fluids and reduce the inconvenience and mess associated with long-term enteral feeding needs would improve the health and quality of life for patients and caregivers. There is currently no product on the market that meets all these needs.

### SUMMARY OF THE INVENTION

The invention is a gastrostomy or nasogastric venting device preferably comprising a vessel having measured gradients along a side surface, and a flexible tube connected to and through a bottom surface. The vessel may be rigid or semi-rigid. The top region of the vessel comprises a gas-permeable covering which can be covered and sealed with a cap member. The cap member can be removed during venting and closed as needed. The flexible tube connected to and through the bottom surface of the vessel is of a length suitable for extending from the vessel. In one embodiment the tubing length is approximately 24 inches and is connectable to a low profile gastrostomy tube, port or connector. In another embodiment, the tubing is directly connectable to a nasogastric tube port or connector. The tube comprises a clamp to allow the tube to be crimped or otherwise closed as needed. The tube may also comprise a connector that permits attachment of an extension set to add additional length to the overall distance from the containment vessel to the gastrostomy or nasogastric connection point. An additional port or connector may be provided at a point distal from the containment vessel, where a feeding set may be attached and liquids for feeding may pass through to the gastrostomy or nasogastric connection point.

The vessel of the venting unit may be a cylindrical syringe-shaped body. The size or volume of the vessel can vary according to the age and size of the patient. Thus, a volume of several ounces would be appropriate for a neonate or infant, while a volume of 8 or more ounces might be more appropriate for an adult. One objective of the invention is to serve as a containment reservoir if a quantity of liquid food is expelled during venting. Thus, a containment vessel of sufficient size to contain at least a portion of a liquid meal is contemplated.

The gas-permeable covering is preferred to be a silicon mesh, but any suitable gas-permeable material can be used, such as a plastic screening or mesh fabric. The covering also serves as a splash-guard to minimize spilling from unexpected or explosive venting that pushes liquids up into the vessel. The mesh may be molded into the device near the top (e.g., ½ inch from the top). Alternatively, the mesh may be associated with the cap, and will preferably be spaced slightly therefrom and be removable.

The cap is open to allow free expulsion of gases during venting. The cap can be closed for ease of transporting the patient from one environment to another, such as moving from a building to a car, from a chair to a bed, for transporting from a hospital room to an X-ray facility, or other transitions, as needed or desired.

The upper region or top of the containment vessel further comprises a connector for connecting the vessel to a support structure. The connector can be a strap, a handle, a clip, an extension of the vessel having a hole, or any other structure that allows the vessel to hang from or attach to the support structure. The support structure may be a pole for hanging IV fluid containers, a chair, an infant seat, a car seat, a bed frame, a hook on a wall or inside an automobile, or a support structure hanging from a ceiling. The support structure may also be a soft surface to which the connector may be pinned, such as the clothing of a caregiver holding a baby or child on their lap.

In one embodiment, the invention is an apparatus and a method for venting gas from the stomach of an individual with a gastrostomy or nasogastric tube. In another embodiment, the invention is an apparatus and a method for venting gas from the stomach while simultaneously providing enteral feeding of an individual with a gastrostomy or nasogastric tube. In yet another embodiment, the invention is an apparatus and a method for relieving gastric pressure in an individual with a gastrostomy or nasogastric tube, wherein the individual is receiving chemotherapy or other treatment that induces nausea, and wherein relief of gastric pressure inhibits or reduces retching and/or vomiting.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIG. 1 illustrates an embodiment of a gastrostomy or nasogastric venting unit.

FIG. 2 illustrates another embodiment of a gastrostomy or nasogastric venting unit, which is connected to an extension set to increase the length of tubing between the containment vessel and the patient.

FIGS. 3A-E show various embodiments of cap and mesh members, including a) a threaded screw cap, b) a snap cap, c) a snap-cap with an O-ring attachment between the cap and vessel, d) a threaded connector securing a mesh to the top of the containment vessel, and e) a mesh molded in the top section of the containment vessel with a cap that fits over the vessel.

FIGS. 4A-C illustrate various embodiments of suitable handles and containment vessels, including a) a woven fabric handle attached to a rigid plastic ring, b) a loop handle extending from an O-ring, and c) a semi-rigid plastic handle attached to a ring.

### DETAILED DESCRIPTION

The invention is a gastrostomy or nasogastric venting unit and methods for its use. Thus, an embodiment of the invention is a pediatric gastrostomy or nasogastric venting device comprising a rigid or semi-rigid vessel defining a specified volume, wherein the vessel has a top region, a bottom region, and one or more sidewalls extending between the top region and the bottom region. Tubing is connected to or connectable to the bottom region of the rigid or semi-rigid vessel, and is connected to or connectable to a gastrostomy tube suitable for gastrostomy stoma or a nasogastric tube. There can be one or more clamps connected or connectable to the tubing at one or more locations between the rigid or semi-rigid vessel and the gastrostomy tube so that the tubing may be clamped if needed.

A splash guard is positioned at the top region of the vessel. The splash guard permits venting but prevents splashing out of material from inside. The splash guard may be molded into the inside surface of the rigid or semi-rigid vessel about, for example, ½ inch from the top. Alternatively, the splash guard may be integrated into a device which can be secured to the top of the vessel where the device has a removable cap, preferably which is spaced away slightly from the splash guard.

The cap can be closed to seal the vessel as needed to temporarily convert the vessel from an open venting system to a closed system, thereby allowing convenient transportation of the device.

Further features include a connector for connecting a support structure such as an IV pole, a handle of a car seat, or other suspending structure.

In one embodiment, the tube connecting to the containment vessel is permanently integrated into the vessel and is about 12-24 inches in length. In another embodiment, the tube is connectable to a commercially available “extension set”. An extension set is used to extend the overall length of the tubing. Exemplary reasons for extending the tubing length include a need to raise the venting unit higher above the patient (thus increasing the amount of pressure needed to vent gases), needing to place the components beyond the reach of the patient, and needing to minimize the apparatus that must be placed near the patient. Extension sets with tubing of various lengths are available with various elements such as a clamp, connectors for administration of medication or feeding, and a connection point at the end of the tube that is suitable for connection to a gastrostomy tube, connector or port.

Extension sets are available in most clinical settings where tube feeding are routinely performed. One such example is a 24″ length Kimberly-Clark® Extension Set and Clamp. Another example is available from Applied Medical Technology. Use of an extension set increases the convenience for home use, where the containment vessel can be washed and reused for 24 hours with a clean extension set, as needed.

Connectors can be traditional Luer-Lock type connectors or any other well-known connecting pieces or adaptors. Unless specialized connector types are required for connection to gastrostomy or nasogastric tube fittings, ENfit® connectors or transition pieces ENFit® adapters and transition pieces (Applied Medical Technology, Inc; Brecksville, Ohio) are good examples of connectors which can be used with this invention.

The containment vessel is preferred to be of a plastic or other unbreakable material. It is an advantage of the invention to be portable, and therefore lightweight materials are preferred. Suitable materials include polyethylene, polypropylene, polystyrene and other rigid or semi-rigid plastics.

In one embodiment, the mesh covering of the upper end of the containment vessel is a silicon mesh. The mesh covering may be molded into the upper end of the containment vessel, for example, ½ inch from the top. In this exemplary embodiment, the mesh would not be removable. In another example, the mesh covering may be secured to the containment vessel with a flexible silicon ring, similar to a rubber band. In another embodiment, the mesh is incorporated into a rigid ring having a diameter greater than that of the containment vessel, and the ring is able to snap over the open end of the containment vessel. In another embodiment, the rigid ring is a cap having mesh-covered hole within a central diaphragm. The size of the mesh-covered hole need only be a few millimeters in order to be adequate for release of gas during venting.

With any configuration of mesh covering, a cap is provided to close over the end of the vessel such that the mesh opening is completely covered and sealed until the cap is removed. In one embodiment, the cap snaps onto the containment vessel. In another embodiment, the cap is threaded and screws onto the containment vessel. The top portion of the containment vessel where the silicone mesh portion is attached, positioned, or molded, may be of a smaller diameter than the threaded portion of the containment vessel, so that the cap surrounds and covers the silicon mesh covering. In one embodiment, the cap is connected to an O-ring that fits around the circumference of the containment vessel to prevent the cap from falling or being lost. In another embodiment, the cap is connected to the containment vessel by a flexible plastic strip able to bend in a hinge-like motion and allow the cap to snap onto the containment vessel. In another embodiment, the cap has a first snapping inner portion with a mesh-covered vent hole and a second snapping outer portion that covers the vent hole and seals the containment vessel when closed. In a further embodiment, the cap simply screws on or is snap connected to the top of the containment vessel, and the mesh is molded into the interior diameter of the containment vessel near the top.

One embodiment is a venting unit that can be used in conjunction with or independently from a feeding set for enteral nutrition. The device permits continuous gas venting before, during or after feeding, since the pressure needed to expel gas through the venting tube is less than that needed to expel gases through a feeding tube when liquid is present. The device comprises a containment vessel for liquids that may be expelled along with the gas. A graduated volumetric scale may be provided on the side of the containment vessel can be used to measure the amount of fluid expelled, thus allowing a caregiver to calculate the actual intake of fluid during feeding. The containment vessel also minimizes spraying, splashing or spilling of gastric fluids that may be expelled during venting at any time.

Another embodiment of the invention is a gastrostomy venting unit that is particularly well-suited for a newborn, neonate or premature infant. The open system and relatively short tube length of the invention requires less pressure than current systems to push gas and any expelled fluids up the tubing of the device. The tube length of 12 to 24 inches is also advantageous because it requires less priming and therefore allows more accurate measurement of liquid nutrition that may be expelled during venting. Due to the low volume of liquids neonates and premature babies are able to consume in one feeding, small and/or sick babies are particularly vulnerable to errors arising from miscalculation of nutritional intake.

Another embodiment of the invention is a method of using the gastrostomy venting unit to immediately relieve gastric pressure and distress in a patient who is receiving chemotherapy or other treatments that induce nausea. When symptoms of nausea are apparent or expected to occur, the gastrostomy venting unit is attached to the gastrostomy tube, connector or port. The open end of the containment vessel is directed downward and below the level of the patient's stomach. The open end can be directed toward a waste container or loosely covered with a disposable diaper, or other disposable paper or container to capture any expelled fluid or gastric contents. In this manner, the patient may avoid the trauma a retching or vomiting. This is particularly advantageous when surgical repairs to the hiatal sphincter, esophagus, or any other upper digestive structures are still in the process of healing.

The invention is advantageous in that it provides a venting unit that can be temporarily capped to prevent accidental spilling or expulsion of liquids during travel or transition from one place to another. A handle on the venting unit can be used to carry it or to suspend it overhead or secure it adjacent to a patient. In the case of a child or infant, the handle can be attached to an infant seat or car seat. Alternatively, it can hang from a hook in a car or other vehicle during travel. When the child is seated in a vehicle and the venting unit is suspended on a hook or other means, the cap may be opened to allow venting to resume.

The size of the containment vessel can vary according to the needs of the patient or individual user. In one embodiment, the containment vessel holds approximately 1-4 ounces, which would typically encompass a meal size for a neonate or young infant. While the primary object of the invention is not focused on providing a vessel for delivering liquid food, it is contemplated that the vessel be sufficient to receive a volume of a meal size that might be expelled when venting gas from the stomach of a patient. Therefore, a vessel size for a larger child or an adult may sometimes need to be larger than one suited for an infant or small child, and individual needs to accommodate explosive venting are also to be considered.

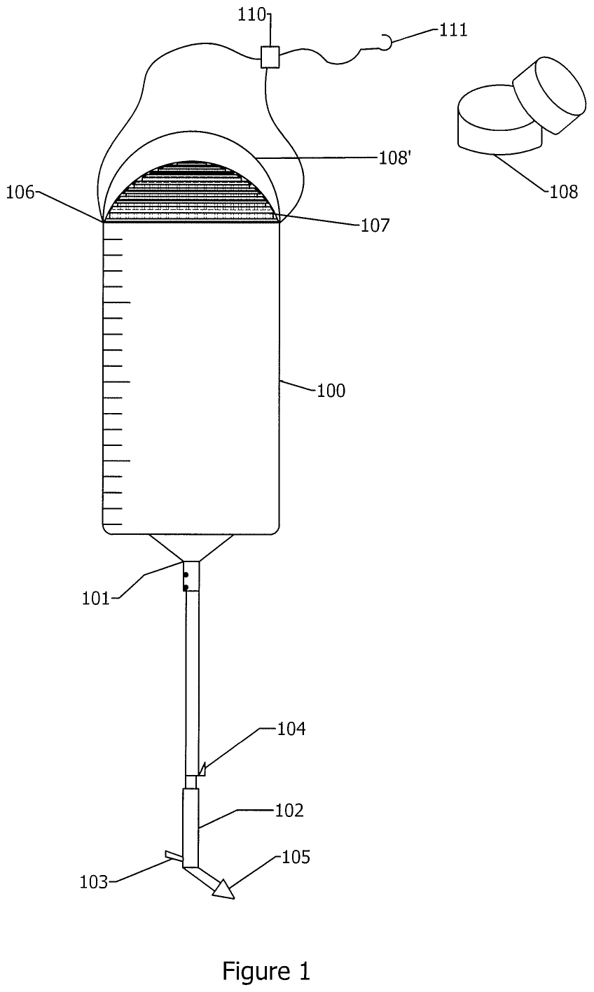

FIG. 1 shows an exemplary venting unit that is particularly well-suited for a neonate or a baby under 2000 grams in weight. The containment vessel 100 can be a cylindrical or other suitable shape in a rigid or semi-rigid plastic. The vessel preferably has markings or raised ridges to indicate the volume of the vessel in ounces, milliliters or both. A bottom surface 101 of the cylindrical vessel is contiguous with a length of clear tubing 102, having a diameter in the range of 12 FR. The tubing length can be in the range of 1 to 36 inches, with a preferred length of approximately 24 inches. Intermediate lengths in the ranges of 6 to 12 or 12-24 inches or more may be desirable for various applications. A feeding set connector 103 is incorporated in the tubing 102 at a point distal from the bottom surface 101, preferably with a position at least 2-4 inches from the terminus of the tubing 102 is preferred. A clamp 104 can be optionally placed at a discretionary point on the tubing 102. The tubing 102 preferably terminates with a cone-shaped connector 105. In some embodiments, the connector may be an L-shaped connector, a Mik-Key® connector, a Mini-One®, or an ENfit® connector type that is able to connect to a gastrostomy tube, conector or port or a nasogastric tube connector or port. In the case of a venting unit for a nasogastric tube, the connector 105 would be one suitable for connecting to a port in a nasogastric tube.

At the top end of the containment vessel 100, a “splash guard”, e.g., silicone mesh 107 may be held in place with a silicone band 106. Alternatively, the silicone mesh 107 may be molded into the inside walls of the containment vessel 100 just below the top of the containment vessel (e.g., about ½ inch below the top. In another embodiment, the silicone mesh 107 may be snap fitted to the top of the containment vessel 100. In still another embodiment, the mesh 107 may be part of a cap structure which is attached to the top end of the containment vessel 100. Different materials may be used for the mesh 107. The primary function of the mesh 107 is to allow gases vented from the subject to pass through, but to contain solid materials from being expelled out the top of the containment vessel 100. The mesh 107 also limits the expulsion of liquids from the containment vessel 100.

In FIG. 1, a cap 108 is shown as being able to snap onto the containment vessel 100 and cover the silicone mesh 107 and silicone band 106. The cap 108 is able to seal the containment vessel 100 when it is in place (shown in shadow form 108′ in FIG. 1), and is easily removed when venting is required. A flexible string or band may encircle the containment vessel 100 just below the silicone band 106 and has string or band extensions 109 on opposite sides of the containment vessel 100 so as to form a handle. Band extensions 109 may be shortened by means of clamp 110. The venting unit may be hung or suspended by the handle or by a hook or other suitable connector 111 at the terminal ends of the strings or bands 109.

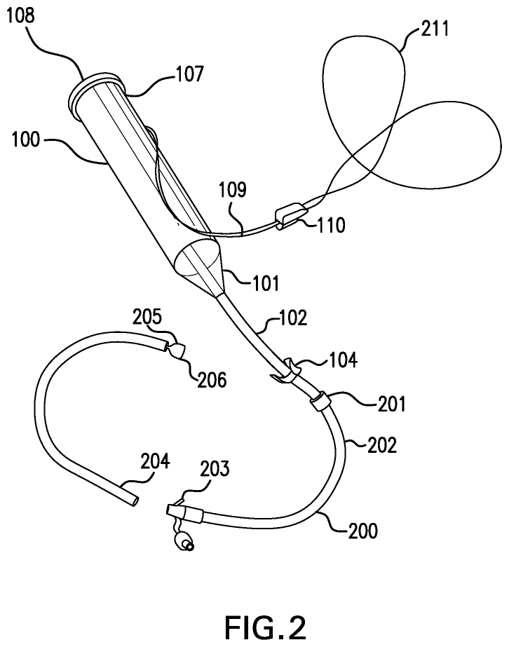

FIG. 2 is an exemplary embodiment of a venting unit that is attached to an extension set to elongate the distance from the bottom surface 101 of the containment vessel 100 to the patient, represented in FIG. 2 by the gastrostomy tube, connector or port (e.g., “button”) 206. Connector 201 can be the attachment point between tubing 102 and extension set 200. Thus, in the illustrated embodiment, tubing 102 terminates in, for example, an ENfit® connector, and extension set 200 comprises a corresponding ENfit® connector piece, and these two pieces meet at connection 201. Tubing 102 preferably comprises a clamp 104. A feeding set connector 203 is incorporated to the end of the clear tubing 202 of extension set 200. The overall length of tubing 202 is preferred to be 12 to 24 inches, with the desired length being dependent upon the needs of a particular patient. Tubing 202 terminates with a connector 205 suitable for connecting to gastrostomy “button” 206. In the case of a venting unit for a nasogastric tube, the connector 205 would be one suitable for connecting to a port in a nasogastric tube. Handle 211 can be a stretch or non-stretch string encircling containment vessel 100 to form a collar, and extending into a loop formation. The loop can be shortened or tightened by adjusting or sliding clamp 110.

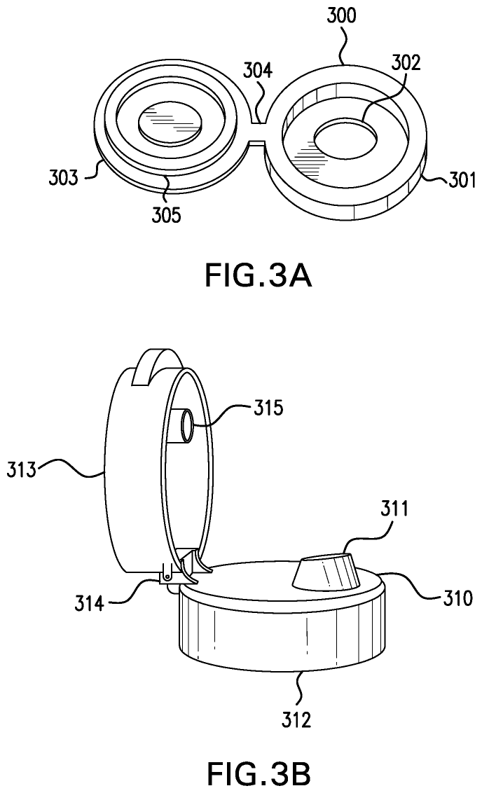

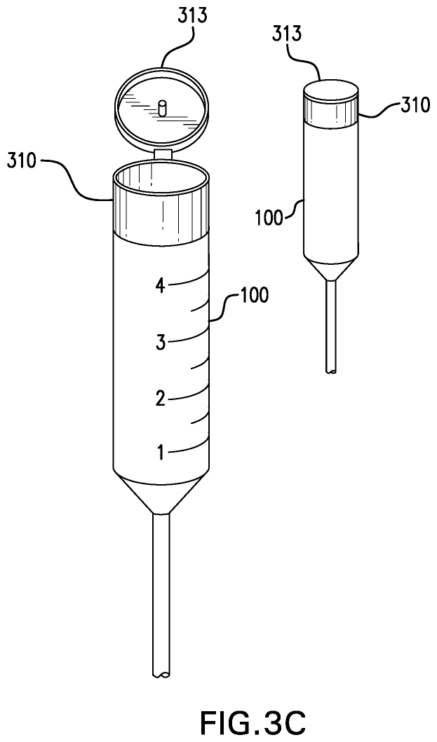

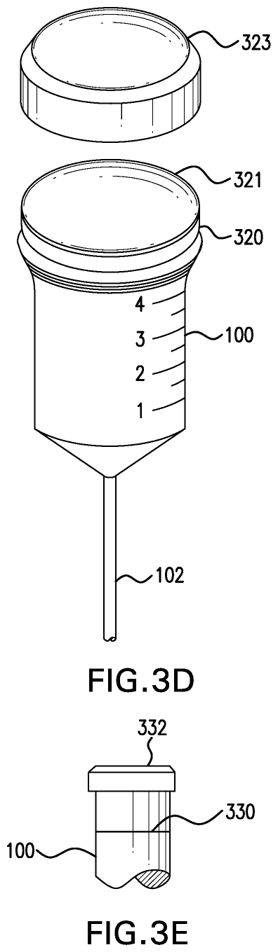

FIG. 3A-E illustrate different examples of caps and meshes that may be used in the practice of the inventions, however many variations of the caps and meshes are possible.

In FIG. 3A, cap 300 comprises a base cap 301 that can be pressed, pushed, or snapped onto the open upper end of a cylindrical containment vessel, such as containment vessel 100 in FIGS. 1 and 2. In one embodiment, cap 300 is made from polypropylene or other semi-rigid plastic material, but other types of plastics and materials, such as metal may also be employed. A mesh fabric 302 is applied to the bottom side of base cap 301, and is visible through a central hole in base cap 301. The mesh fabric may be silicone or other suitable materials and, as described above, is designed to allow gases expelled from the patient to pass during venting, but to contain solids and possibly to contain or slow down the fluids expelled from the patient. A sealing cap 303 is connected with flexible hinge 304 to base cap 301. When sealing cap 303 is folded over base cap 301, the two can snap together and seal by pressing sealing cap 303 so that sealing ring 305 is below the upper lip of base cap 301.

In FIG. 3B, the base cap 312 is a threaded screw-on cap that corresponds to a threaded upper end of a containment vessel. A mesh fabric or other filtering material is applied to the inner surface 312 of base cap 310. Venting occurs through vent 311 on the upper surface of base cap 310. Sealing cap 313 is attached to base cap 310 by a flexible hinge 314. When sealing cap 313 is depressed over base cap 310, protrusion 315 fits and snaps into vent 311 and provides a closed cap that is easily reopened by lifting sealing cap 313.

In FIG. 3C, the base cap 310 is on a containment vessel 100 with sealing cap 313 in the open (left) and venting position, and in the closed (right) and sealed positions.

In FIG. 3D, the containment vessel 100 has a screw-on cap 320 comprising a mesh 321 covered opening and slip-on cap 323 that is large enough to cover screw-on cap 320, but fits snugly for transitioning from one location to another.

In FIG. 3E, the mesh 330 is molded inside the top end of the containment vessel 100, preferably ½ inch from the top, and a cap 332 is able to be fitted over the top of the containment vessel 100. The cap 332 can be connected to the vessel 100 by a hinge, or be snap fitted or screw fitted over the top of the containment vessel 100.

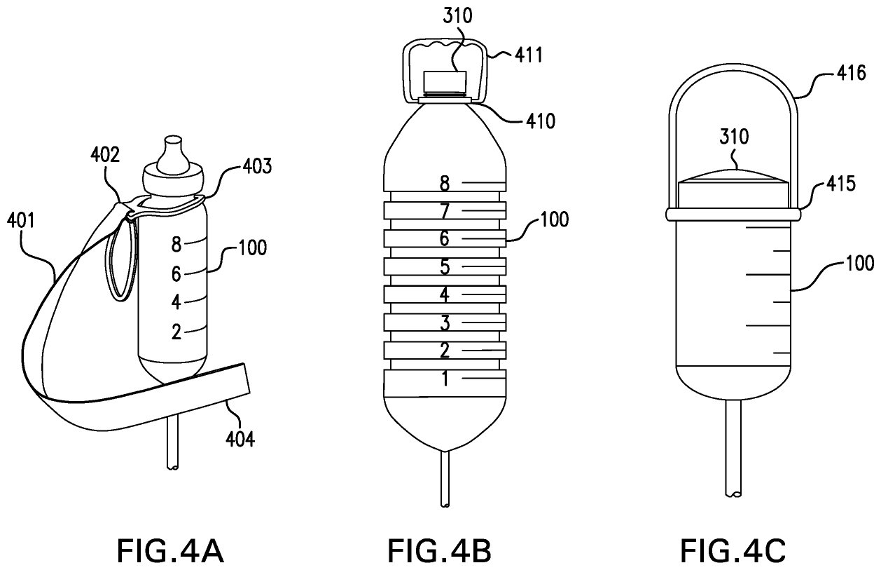

FIGS. 4A-C illustrate various examples of handles and containment vessels that may be used with the invention.

FIG. 4A shows a woven solid material is used to form strap 401. The strap 401 is looped to a ring 403 or a component of ring 403 and sewn to form an attachment 402. Ring 403 is a size suitable for a vessel 100 positioned below a cap 310. A second loop 404 is formed at the distal end of strap 401 by sewing, or applying closing material such as VELCRO™. The second loop 404 may be opened and closed around a support structure, or applied to a matching closing material. The containment vessel 100 is shown as a bottle shape with indicia on the side.

FIG. 4B shows a semi-rigid plastic ring 410 placed over the mouth of a containment vessel 100 and secured in place when a screw-on cap 310 is assembled onto the containment vessel 100. A flexible semi-rigid handle 411 is attached to opposite sides of semi-rigid ring 410. The containment vessel 100 is bottle shaped and includes ridges and/or indicia on its outer surface.

FIG. 4C shows an elastic band 415 stretched and applied around containment vessel 100 below cap 310. An elastic loop handle 416 extends from opposite sides of elastic band 415. Elastic loop handle 416 can be hung from a hook support or pinned to a soft surface support. The containment vessel 100 is syringe shaped in FIG. 4C, but any desirable shape may be employed so long as it is constructed from a rigid or semi-rigid material.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.). . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

These Examples describe materials and methods for using embodiments illustrated in FIGS. 1-4; additional details can be found above under “Brief Description of the Drawings”.

### Example 1

A venting unit comprising a containment vessel having a volume of 4 ounces and a tubing length of 24 inches is attached to a gastrostomy tube on a baby weighing 1500 grams. The venting unit is suspended at about 6 inches above the level of the baby's stomach. The cap is unsealed to allow venting. A feeding set is attached to the feeding set port and feeding with a liquid infant formula begins at a variable rate dependent on weight. Venting is observed to be inadequate, and needs to be increased. The venting unit is lowered in increments of about 1 inch until venting is observed to be adequate. When liquid formula enters the containment vessel, the volume is noted and deducted from the total intake at the end of the feeding. Venting is allowed to continue after feeding, until baby is to be moved to allow mother to hold baby. The containment vessel is capped and baby is moved and positioned in mother's arms. When motion is completed, the venting unit is resuspended at about 12 inches over the baby's stomach by pinning the handle to the mother's clothing.

### Example 2

A venting unit is attached to an extension set by means of ENfit® connecting pieces. The total length of tubing is 24 inches in length. The venting unit is attached to a nasogastric tube of a young baby, and the cap is opened to allow venting. The venting unit is suspended approximately 12 inches over the baby's stomach. A feeding set is attached and liquid nutrition is supplied through the delivery tube. The delivery rate is well-tolerated and is increased. The venting rate is adequate, and the unit can be raised approximately 6 inches in assistance with the increased rate of liquid delivery.

### Example 3

A baby is recovering from a surgical procedure and is beginning to tolerate liquid feeding and has less dependency on the venting unit. The venting unit is capped for short periods after enteral feeding to help the baby learn to tolerate the distention of the stomach. If gagging or retching is prolonged (e.g., greater than 1-2 mins) the cap is reopened to allow venting.

### Example 4

A young child with a gastrostomy is discharged from the hospital and is still in need of enteral feeding and venting. The child is able to tolerate car rides when the venting unit is opened to vent and suspended from the coat hook in the car next to her car seat. The parent can close the cap on the venting unit when removing the child from the car seat and transitioning to a stroller. The venting unit can be suspended from the stroller cover and reopened to allow venting. The containment vessel is of a sufficient volume so that unexpected venting that is a mixture of gastric fluids and gases does not explode inside the car or at the destination location.

### Example 5

A young child has undergone a surgical reconstruction of the upper digestive tract as a result of cancer. A gastrostomy is performed along with the cancer surgery to allow time for healing of the reconstructed areas. A venting unit is connected to the gastrostomy button and remains in place at all times during the healing process. In conjunction with the surgery, the child is receiving chemotherapy treatment that induces nausea. The nausea is immediately relieved when a caregiver holds the open venting unit below the level of the child's stomach. The gastric contents can be collected in a bedside waste container, emesis basin, disposable diaper or other paper products.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1-15. (canceled)

16. A method for allowing for venting gas while inhibiting loss or spilling of gastric fluid or material when transporting a patient having a gastrostomy or nasogastric tube, comprising:
attaching an open venting unit to said gastrostomy or nasogastric tube, wherein said open venting unit comprises a cap that can be securely sealed temporarily,
closing said cap to seal the open venting unit to form a closed venting unit,
transferring said patient from a starting location to a secondary location,
hanging the closed venting unit at a height above the stomach of the patient, and
opening said cap to allow venting by the open venting unit.

17. A method for relieving nausea and avoiding retching or vomiting in a patient having a gastrostomy or nasogastric tube, comprising:
attaching an open venting unit to said gastrostomy or nasogastric tube,
capturing gastric materials from a stomach of the patient, while the open venting unit is attached to said gastronomy or nasogastric tube while permitting venting of gas through said materials and out an open top of said open venting unit,
periodically positioning the open venting unit below the patient's stomach,
pointing an open top of the open venting unit downward when said open venting unit is positioned below the patient's stomach to empty the gastric materials,
wherein said periodically positioning and pointing step are performed a plurality of times while said open venting unit is attached to said gastrostomy or nasogastric tube so as to relieve nausea and avoid retching or vomiting by said patient.

18. The method of claim 17 further comprising capturing the gastric materials in a container while the open top of the open venting unit is pointed downward.

19. The method of claim 17 wherein the open venting unit is comprised of a rigid or semi-rigid vessel defining a specified volume, wherein the rigid or semi-rigid vessel has a top region, a bottom region, and one or more sidewalls extending between the top region and the bottom region, wherein the top region has a first opening which is approximately as wide as the rigid or semi-rigid vessel, wherein the bottom region has a second opening that is smaller than the first opening, and wherein the second opening is designed to pass gasses and fluid from the stomach of a patient into the rigid or semi-rigid vessel, and the first opening is designed to pass the gasses into an external environment during,
wherein gases are passed into the external environment during said capturing step.

20. The method of claim 19 wherein the open venting unit further comprises a splash guard positioned at said top region of said rigid or semi-rigid vessel, wherein said splash guard permits venting but prevents splashing out of material from inside said specified volume through said first opening.

21. The method of claim 16 wherein the open venting unit is comprised of a rigid or semi-rigid vessel defining a specified volume, wherein the rigid or semi-rigid vessel has a top region, a bottom region, and one or more sidewalls extending between the top region and the bottom region, wherein the top region has a first opening which is approximately as wide as the rigid or semi-rigid vessel, wherein the bottom region has a second opening that is smaller than the first opening, and wherein the second opening is designed to pass gasses and fluid from the stomach of a patient into the rigid or semi-rigid vessel, and the first opening is designed to pass the gasses into an external environment,
wherein said cap fits over said first opening when sealing the opening venting unit, and
wherein venting during said opening the cap step occurs through the first opening.

22. The method of claim 21, wherein the open venting unit further comprises a splash guard positioned at said top region of said rigid or semi-rigid vessel, wherein said splash guard permits venting but prevents splashing out of material from inside said specified volume through said first opening.
